(19) United States
(12) Patent Application Publication
(10) Pub. No.: US 2026/0298895 A1
(43) Pub. Date: Oct. 1, 2026

(54) CHLORIDE MEASUREMENT WITH SILVER, 2,4,6-TRIPYRIDYL-S-TRIAZINE AND IRON (II) TITRATION

(71) Applicant: Hach Company, Loveland, CO (US)

(72) Inventor: Devin Lee Maurer, Ankeny, IA (US)

(21) Appl. No.: 19/094,291

(22) Filed: Mar. 28, 2025

(51) Int. Cl. G01N 31/16; G01N 21/78

(52) U.S. Cl. CPC G01N 31/16; G01N 21/78

(57) ABSTRACT

An embodiment provides a method for measuring an amount of chloride in a sample, including: adjusting the pH of the sample to an acidic pH; introducing an amount of silver nitrate and an amount of iron (II) to the sample to make a reaction solution, wherein the amount of silver nitrate is in excess of the amount of chloride; back titrating the reaction solution using a 2,4,6-Tripyridyl-s-triazine (TPTZ) solution; or the TPTZ could be introduced to the reaction solution up front with the silver nitrate and iron (II); back titrating the reaction solution with a chloride solution; or introducing only an amount TPTZ and an amount of iron (II) to the sample to make a reaction solution; titrating the reaction solution using a silver nitrate solution; and determining the amount of chloride in the sample from the amount of titrant used to reach the titration endpoint. Other aspects are described and claimed.

### BACKGROUND

This application relates generally to measuring chloride in a sample, and more particularly, to measuring chloride in the presence of 2,4,6-Tripyridyl-s-triazine (TPTZ), silver nitrate, and iron (II).

Ensuring water quality is critical in a number of industries such as pharmaceuticals and other manufacturing fields. Additionally, ensuring water quality is critical to the health and well-being of humans, animals, and plants which are reliant on the water for survival. Analytes such as chloride may be measured and monitored. Chloride levels outside of acceptable parameters in water can be harmful to humans or animals, or interfere with proper disinfection or treatment of water. For example, chloride may cause the water to be less desirable to consumers or facilities. Chloride may be present from natural or human activities such as manufacturing. Measurement and mitigation of chloride may result in higher costs of water treatment. Therefore, detecting the presence and concentration of chloride in water or other liquid solutions is vital.

### BRIEF SUMMARY

In summary, one embodiment provides a method for measuring an amount of chloride in a sample, comprising: adjusting the pH of the sample to an acidic pH; introducing an amount of silver nitrate and an amount of iron (II) to the sample to make a reaction solution, wherein the amount of silver nitrate is in excess of the amount of chloride; back titrating the reaction solution using 2,4,6-Tripyridyl-s-triazine (TPTZ) solution; and determining the amount of chloride in the sample from the amount of the titrant used to reach the titration endpoint.

Another embodiment provides a method for measuring an amount of chloride in a sample, comprising: adjusting the pH of the sample to an acidic pH; introducing an amount of silver nitrate, 2,4,6-Tripyridyl-s-triazine (TPTZ), and an amount of iron (II) to make a reaction solution, wherein the amount of silver nitrate is in excess of the amount of chloride; back titrating the reaction solution using a chloride solution; and determining the amount of chloride in the sample from the amount of the titrant used to reach the endpoint.

A further embodiment provides a method for measuring an amount of chloride in a sample, comprising: adjusting the pH of the sample to an acidic pH; introducing an amount of 2,4,6-Tripyridyl-s-triazine (TPTZ) and an amount of iron (II) to the sample to make a reaction solution; titrating the reaction solution using a silver nitrate solution; and determining the amount of chloride in the sample from the amount of the titrant used to reach the endpoint.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a back titration using silver in combination with TPTZ and iron (II) to measure chloride.

FIG. 2 illustrates another back titration using silver in combination with TPTZ, iron (II) and chloride to measure chloride.

FIG. 3 illustrates a forward titration using silver in combination with TPTZ and iron (II) to measure chloride.

FIG. 4 illustrates a flow diagram of an example chloride measurement method.

FIG. 5A illustrates a Hach® mercuric thiocyanate colorimetric method to measure chloride.

FIG. 5B illustrates a Hach® QuanTab test strip method to measure chloride.

FIG. 5C illustrates a Hach® silver chloride turbidity method to measure chloride.

FIG. 5D illustrates a Hach® silver nitrate dichromate titration method to measure chloride.

FIG. 6A illustrates a Hach® mercuric nitrate diphenylcarbazone titration method to measure chloride.

FIG. 6B illustrates a mercuric TPTZ ferrous colorimetric method to measure chloride.

FIG. 7A illustrates a silver thiocyanate titration method to measure chloride.

FIG. 7B illustrates a silver dichlorofluorescein titration method to measure chloride.

FIG. 8 illustrates example data of the color change approaching and after the endpoint of the titration of a 40 mg/L chloride sample using silver in combination with TPTZ and iron (II) to measure chloride compared to other existing methods.

FIG. 9 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Chloride measurement in water or other aqueous samples or solutions is important for many different reasons. For example, chloride measurement may be used to determine the quality of water. High concentrations may be harmful to animals, humans, and/or plants. Accordingly, as another example, a user or entity may want the chloride in a body of water to be under a particular threshold, therefore, the user may measure the chloride in order to determine if the amount of chloride is under that threshold. Chloride may be present in a body of water either naturally or from human activity such as manufacturing or storage conditions. Also, a level of chloride may need to be monitored and/or controlled in a solution for human consumption, medical, industrial, food/beverage, or manufacturing applications. As another example, chloride may be introduced into a sample for disinfection or preparation of a volume of water for human consumption, food manufacturing, pharmaceutical manufacture, or the like.

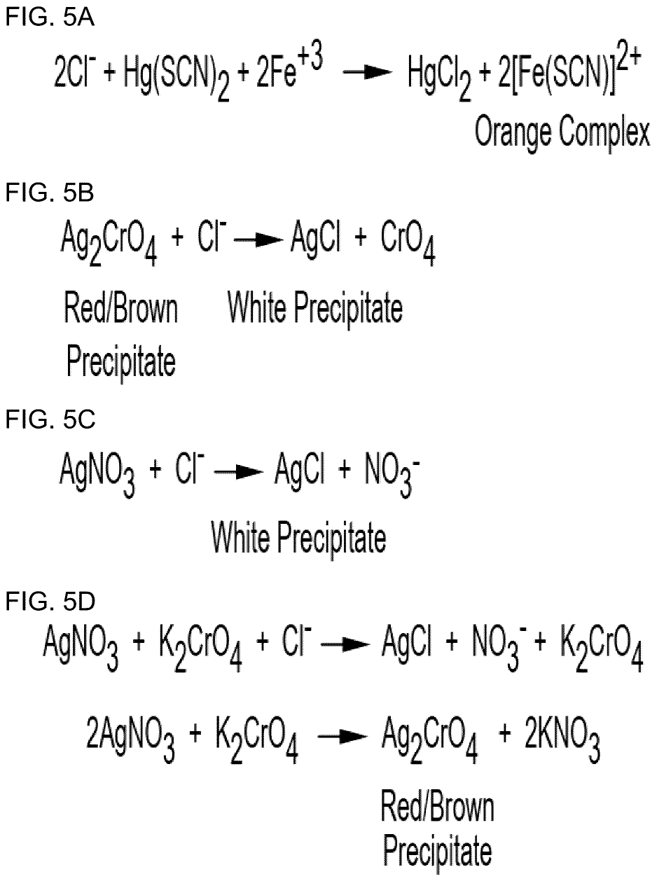

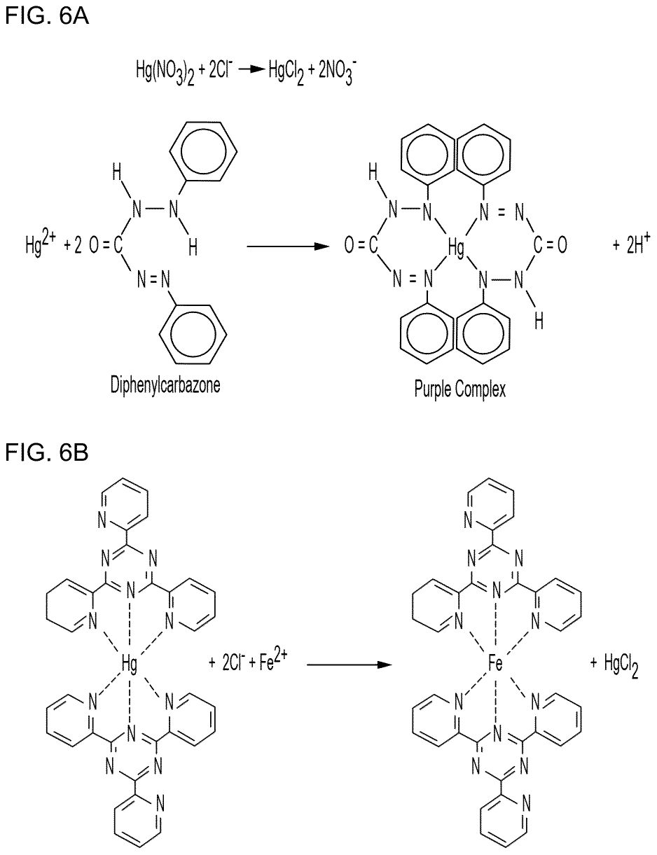

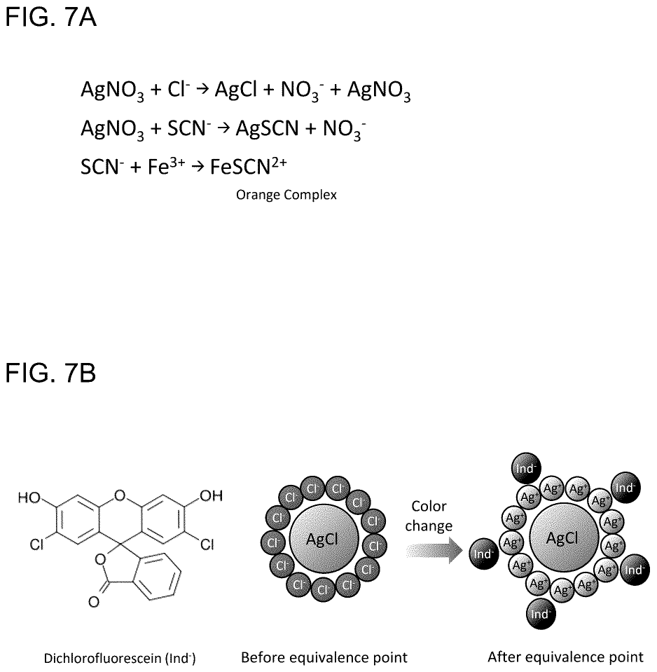

Conventional methods of chloride measurement and detection may have limitations discussed herein. For example, conventional methods may use mercury, dichromate, and/or thiocyanate. Governments may have limits of use or waste disposal requirements for this conventional method. Such reagents pose a hazard to both a user and remain a costly step to properly dispose of the reagents. Various methods using these harmful reagents are illustrated in FIG. 5, FIG. 6, and FIG. 7. Such examples include mercuric thiocyanate colorimetric method (FIG. 5A), QuanTab tests strip method (FIG. 5B), silver nitrate dichromate titration method (FIG. 5D), and mercuric nitrate Diphenylcarbazone titration method (FIG. 6A), available from Hach Company, Loveland CO, USA (HACH is a registered trademark of Hach Company in the United States and other countries). Further examples of chloride measurement may include a silver chloride turbidity method (FIG. 5C), available from Hach Company, Loveland CO, USA (HACH is a registered trademark of Hach Company in the United States and other countries, mercuric TPTZ ferrous (FIG. 6B), silver thiocyanate method (FIG. 7A), and silver dichlorofluorescein method (FIG. 7B).

However, there are some limitations with these methods. Mainly, the methods use mercury, dichromate, and/or thiocyanate. At the very least, such reagents may cause irritation of the skin, eyes, nose, and throat. The reagents are also known carcinogenic substances. Therefore, the reduction or complete elimination of the use of these reagents is beneficial to users of a product, and reduces cost, storage, and disposal costs for entities using the product. Current methods, systems, and kits for chloride measurement using the above method involve hazardous or controlled reagents make obtaining the reagents and disposal difficult. What is needed is an accurate method to measure chloride in a sample with less hazardous reagents and more accuracy at low concentrations of chloride.

Accordingly, an embodiment provides a method for measuring chloride using silver nitrate, 2,4,6-Tripyridyl-s-triazine (TPTZ), and iron (II). In an embodiment, hazardous reagents such as mercury, dichromate, and/or thiocyanate alone or in combination are not used unlike conventional methods. Chloride may be measured using various methods. A first method introduces an amount of silver nitrate and an amount iron (II) to the sample to make a reaction solution, wherein the amount of silver nitrate is in excess of the amount of chloride, and back titrates the reaction solution using an amount of 2,4,6-Tripyridyl-s-triazine (TPTZ) solution. A second method introduces an amount of silver nitrate, 2,4,6-Tripyridyl-s-triazine (TPTZ), and an amount iron (II) to make a reaction solution, wherein the amount of silver nitrate is in excess of the amount of chloride, and back titrates the reaction solution using an amount of chloride solution. A third method introduces 2,4,6-Tripyridyl-s-triazine (TPTZ) and an amount of iron (II) to the sample to make a reaction solution and titrates the reaction solution using an amount of silver nitrate solution. In an embodiment, an auto titrator equipped with a photometric probe designed to detect the endpoint of the above embodiments may be used to measure an amount of chloride in an aqueous sample or solution in an automated version. The aqueous sample may contain an amount of chloride to be measured. In an embodiment, the reaction or indicator reagents may be introduced to the aqueous sample in a form selected from the group consisting of: a solution, a powder, and a prepackaged module. In an embodiment, the aqueous sample may be a sample of water for quality testing. The system and method may use the eye or photometric probe to determine the endpoint of the titration.

The illustrated example embodiments will be best understood by reference to the figures. The following description is intended only by way of example, and simply illustrates certain example embodiments.

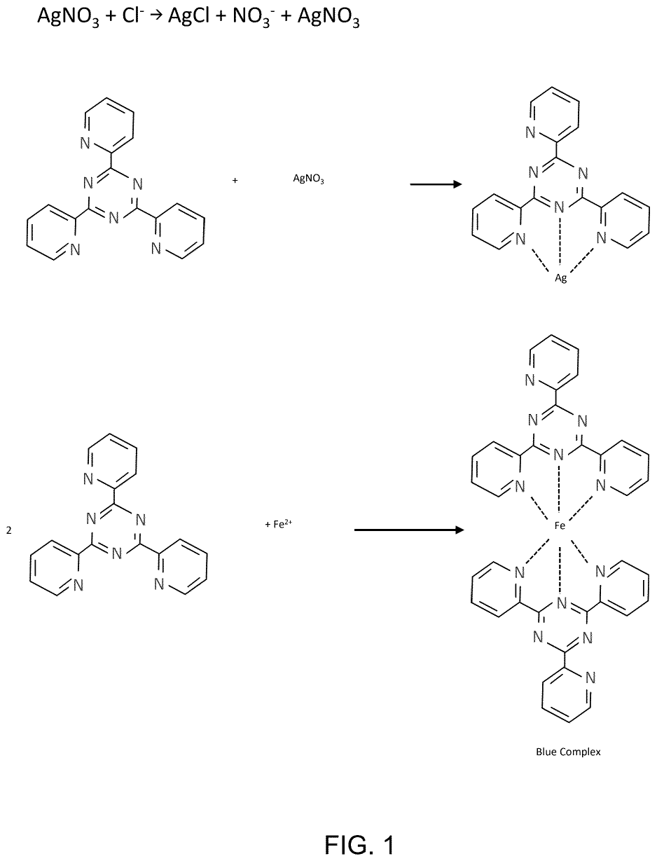

Referring to FIG. 1, in an embodiment, a reaction schematic is illustrated using silver nitrate, 2,4,6-Tripyridyl-s-triazine (TPTZ) and iron (II) for the detection and measurement of chloride. In this example, a known amount of silver, in excess to the working chloride range of the test, and iron (II) are added to the sample. The excess silver is then back titrated with TPTZ. In other words, a known amount of silver nitrate in excess to an amount of chloride in the sample, may be back titrated with a TPTZ solution in the presence of iron (II). The back titration may be performed under acidic conditions to an endpoint in which the solution has a blue color. The pH may be adjusted to an acidic condition using acetate buffer. The acidic condition may be defined as a pH range of 4.0 to 5.5. The silver nitrate in combination with TPTZ and iron (II) to determine chloride with a titration test or method that could be utilized at the bench or in the field without the use of hazardous chemical such as mercury, thiocyanate, or dichromate as used in other chloride tests.

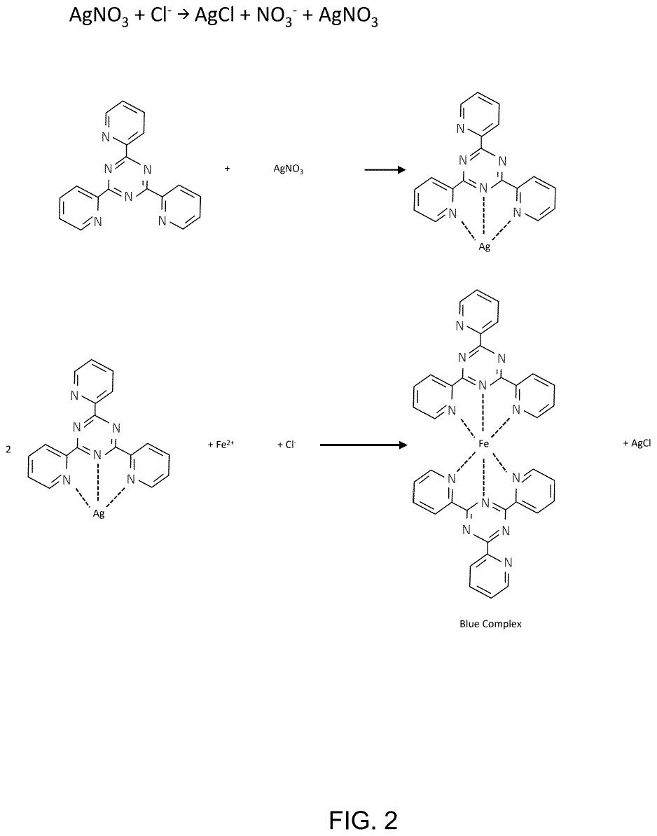

Referring to FIG. 2, in another embodiment, a reaction schematic is illustrated using silver nitrate, 2,4,6-Tripyridyl-s-triazine (TPTZ), iron (II) and chloride for the detection and measurement of chloride. In this example, a known amount of silver, in excess to the working chloride range of the test, TPTZ and iron (II) are added to the sample. The excess silver is then back titrated with chloride. In other words, a known amount of silver nitrate in excess to an amount of chloride in the sample, may be back titrated with a chloride solution in the presence of TPTZ and iron (II). The back titration may be performed under acidic conditions to an endpoint in which the solution has a blue color. The pH may be adjusted to an acidic condition using acetate buffer. The acidic condition may be defined as a pH range of 4.0 to 5.5. The silver nitrate in combination with TPTZ, iron (II) and chloride to determine chloride with a titration test or method that could be utilized at the bench or in the field without the use of hazardous chemical such as mercury, thiocyanate, or dichromate as used in other chloride tests.

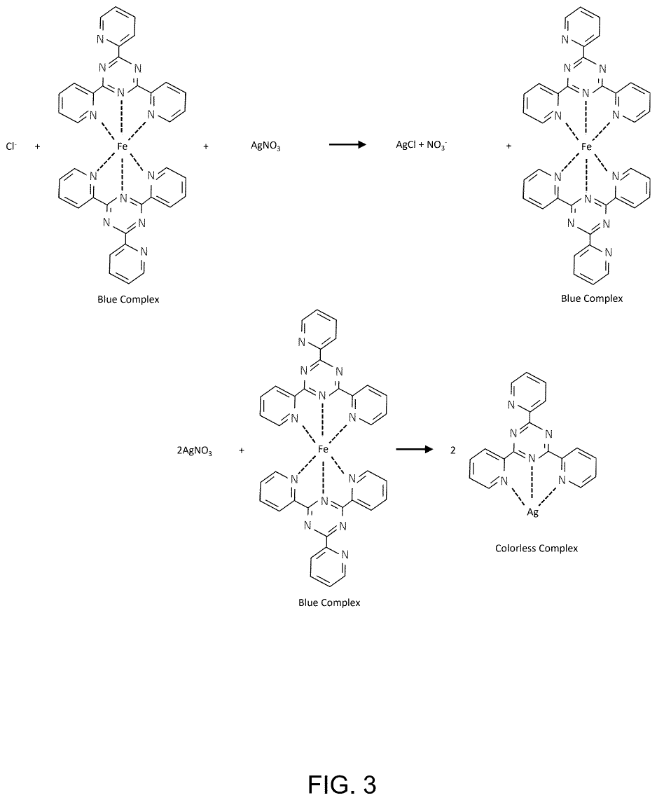

Referring to FIG. 3, in a further embodiment, a reaction schematic is illustrated using silver nitrate, 2,4,6-Tripyridyl-s-triazine (TPTZ) and iron (II) for the detection and measurement of chloride. The method may use a forward titration. In this example, an amount of TPTZ and iron (II) are added to the sample. The chloride is then titrated with silver nitrate. The titration may be performed under acidic conditions to an endpoint in which the solution has a colorless or near colorless (yellow) appearance. The pH may be adjusted to an acidic condition using acetate buffer. The acidic condition may be defined as a pH range of 4.0 to 5.5. The silver nitrate in combination with TPTZ and iron (II) to determine chloride with a titration test or method that could be utilized at the bench or in the field without the use of hazardous chemical such as mercury, thiocyanate, or dichromate as used in other chloride tests.

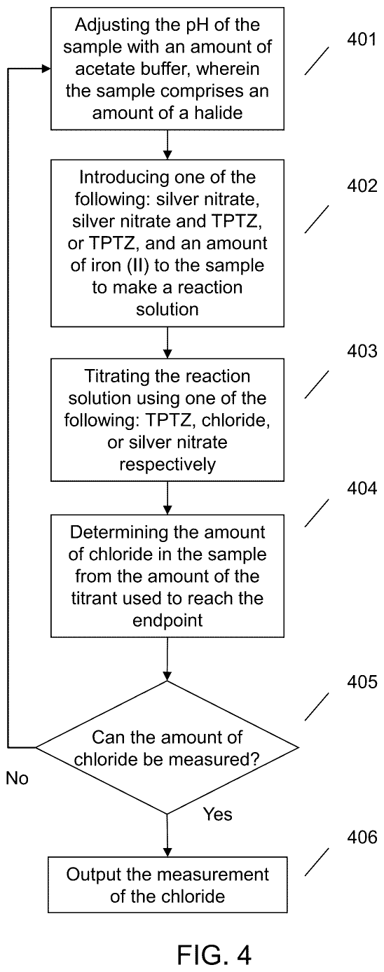

Referring to FIG. 4, in an embodiment, an example method and system for measurement of chloride in a sample or an aqueous sample or solution is illustrated. The method of titration may be selected from the different titration methods disclosed herein.

The aqueous sample may include a sample from a natural body of water, a holding tank, a processing tank, a pipe, industrial effluent, wastewater, or the like. The solution may be in a continuous flow, a standing volume of liquid, or any combination thereof. In one embodiment, the solution may be introduced to a buffer, for example, in a test chamber of the measurement device. In an embodiment, the measurement device may be a benchtop, field, or hand-held device. A hand-held device may have advantages such as lower cost, portability, field use, or the like. Introduction of the solution into the measurement device may include placing or introducing the solution into a test chamber manually by a user or using a mechanical means, for example, gravity flow, a pump, pressure, fluid flow, or the like. For example, a water sample for chloride measurement may be introduced to a measurement or test chamber using a pump. In an embodiment, valves or the like may control the influx and efflux of the solution into or out of the one or more chambers, if present.

A chamber, vessel, cell, or the like may contain an aqueous sample and associated reagents. Various reagents may be added to an aqueous sample in the form of a powder, a liquid, a prepackaged module, or the like. A device may contain one or more bottles of reagents which contain necessary reagents. The reagents contained in the one or more bottles may be pump fed or gravity fed. The flow of the reagents may be metered to ensure proper volume delivery to the measurement cell. The aqueous sample may be fed through a pressured inlet, a vessel, or the like. The aqueous sample may be introduced into the measurement chamber by a pump or gravity fed. The sampling device may be in series or parallel to an aqueous flow. The device may have a system to ensure proper mixing of the aqueous sample with a reagent. The method or device may have a heating element to heat a sample and/or reagents.

Additionally or alternatively, the measurement device may be present or introduced in a volume of the solution. The measurement device may then be exposed to the volume of an aqueous sample where it may perform measurements. The system may be a flow-through system in which an aqueous sample and/or reagents are automatically mixed and measured. Once the sample is in contact with the measurement system, the system may measure the chloride or an amount of chloride of the sample, as discussed in further detail herein. In an embodiment, the measurement device may include one or more chambers in which the one or more method steps may be performed.

In an embodiment, a sample with chloride to be measured may be a predetermined volume or brought to a predetermined volume with deionized water. Reagents described herein may be added to this volume. There may be more than one reagent added at different points in the measurement process. In other words, a first reagent may be added, the sample mixed, then a second reagent added, and then mixed once again. Mixing may be accomplished by mechanical inversion, stirring, pumping, or the like. Mixing may be performed at intermediate steps in between the addition of different reagents.

Referring to FIG. 4. at 401, in an embodiment, the sample with an amount of chloride may be adjusted to an acidic pH. The pH adjustment may be performed by adding an amount of acetate buffer to the aqueous sample or sample comprising an amount of chloride. The acidic pH may be in the range of about pH 4.0 to 5.5.

In an embodiment, the aqueous sample may be buffered or adjusted to a pH value. In an embodiment, a pH value may be selected to minimize interferences. For example, a pH may be selected based upon the concentration and/or composition of interferences. In an embodiment, the sample or reaction solution may be pH adjusted to 4.0-5.5 using acetate buffer. The buffer, like any reagent, may be added by hand, gravity fed, or pumped using associated valves, tubing, or the like. The addition of reagents may be controlled by a system or product as described herein.

At 402, in an embodiment, one or more reagents may be added to the sample with an amount of chloride to be measured. Depending on which titration method is to be used different reagents may be added. The reagents may include silver nitrate, 2,4,6-Tripyridyl-s-triazine (TPTZ), and iron (II) may be added to the sample with the acetate buffer which may be referred to as the pH adjusted sample solution or a reaction solution.

At 403, in an embodiment, a titration or back titration may be performed. For example, silver nitrate may be added to a sample and back titrated with a TPTZ solution in the presence of iron (II) to a blue endpoint under acidic conditions. The silver nitrate is added in excess of the amount of chloride in the sample. As another example, silver nitrate may be added to the sample and back titrated with a chloride solution in the presence of TPTZ and iron (II) to a blue color endpoint under acidic conditions. In a further example, a chloride sample may have TPTZ added and titrated with silver nitrate in the presence of iron (II) to a colorless to yellow endpoint from blue solution under acidic conditions. An amount of chloride in the sample or reaction solution may be determined or calculated based upon how much titrant is required for a given titration or back titration.

In an embodiment, a sample or aqueous sample may be prepared. Reagents for the indicator and buffer, may be placed in a solution, aqueous sample, water sample or the like. The sample may be added, with other components, to a chamber, vessel, or the like as a powder, a liquid, or a prepackaged module. The sample and/or components may be added manually or using an autonomous system. In other words, the reagents for the method may be prepackaged and/or premeasured for ease of use. The prepackaged reagents may be added to a sample, or the sample may be added to a prepackaged reagent container or vial. Therefore, the silver nitrate, TPTZ, buffer and iron (II) may be in prepackaged modules or a plurality of prepackaged modules for combination with a sample with the chloride to be measured.

At 404, in an embodiment, an amount of chloride may be measured using a titration method. The chloride may be present in the reaction solution. In an embodiment, the presence of chloride in an aqueous solution may cause a color change signaling the endpoint of the titration. Accordingly, a measurement device or user can correlate the measured endpoint of the titration with the amount of chloride in the aqueous sample based off the amount of titrant used to reach the endpoint.

At 405, in an embodiment, the system and method may determine if an amount of chloride measured is valid. For example, an amount of chloride may be measured using titration methods by hand or automated. The calculated values may be compared to expected values, historical values, or the like. Chloride measurement using titration methods may be at periodic intervals set by the user or preprogrammed frequencies in the device. Measurement of chloride by a device allows for real time data with very little human involvement in the measurement process. In the event that the system outputs an unexpected value, the system may automatically request re-measurement of a solution or sample or continue with titration.

A programmed calibration curve may be entered into the device for calibrating the measurement device. In an embodiment, the system and method may be periodically tested using a known amount of chloride in the sample. The system may then recalibrate or send an error report for maintenance. In the event that the error is caused by an unclean device or that the device otherwise needs cleaned, the system may implement a cleaning cycle. Cleaning of the photometric probe or measurement chamber may be required at an unspecified time interval, after a certain number of measurements, upon user or system request, or the like. In an embodiment, a cleaning cycle of the measurement device may be performed using either automated or manual methods.

At 405, in an embodiment, if a concentration of chloride or amount of chloride cannot be determined, the system may continue to measure chloride. Additionally or alternatively, the system may output an alarm, log an event, or the like. If a concentration of chloride can be determined, the system may provide a measurement of chloride concentration at 406. The measurement which may be the titrant amount or chloride concentration may be an output that is provided to a device in the form of a display, printing, storage, audio, haptic feedback, or the like. Alternatively or additionally, the output may be sent to another device through wired, wireless, fiber optic, Bluetooth®, near field communication, or the like.

An embodiment may use an alarm to warn of a measurement or concentration outside acceptable levels. An embodiment may use a system to shut down water output or shunt water from sources with unacceptable levels of chloride. For example, a chloride measuring device may use a relay coupled to an electrically actuated valve, or the like. The system may connect to a communication network. The system may alert a user or a network. This alert may occur whether a chloride measurement is determined or not. An alert may be in a form of audio, visual, data, storing the data to a memory device, sending the output through a connected or wireless system, printing the output or the like. The system may log information such as the measurement location, a corrective action, geographical location, time, date, number of measurement cycles, or the like. The alert or log may be automated, meaning the system may automatically output whether a correction was required or not. The system may also have associated alarms, limits, or predetermined thresholds. For example, if a chloride concentration reaches a threshold. Alarms or logs may be analyzed in real-time, stored for later use, or any combination thereof.

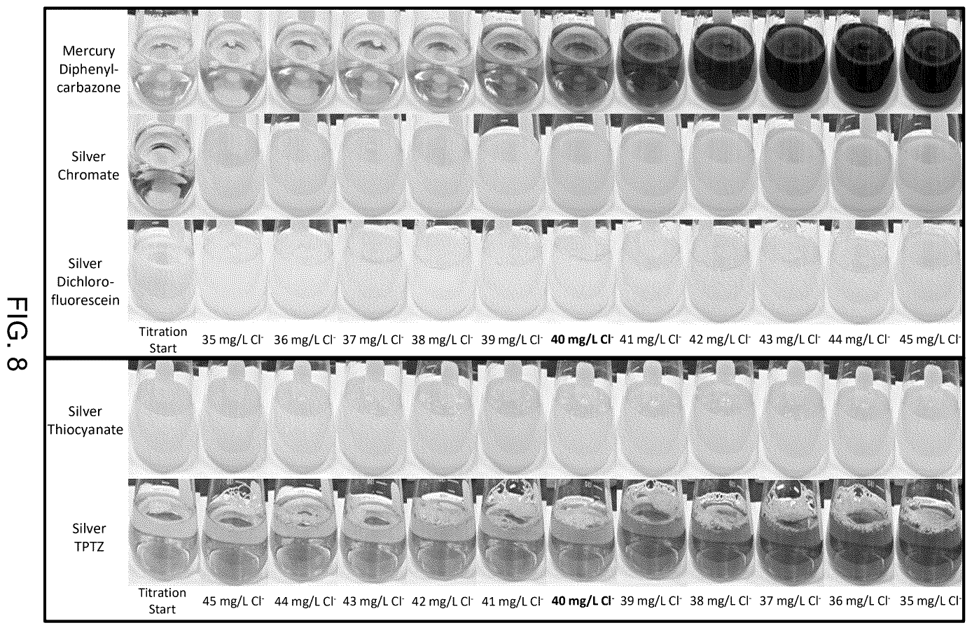

Referring to FIG. 8, in an embodiment, example data are illustrated using mercury diphenylcarbazone, silver chromate, and silver dichlorofluroescein forward titration methods on a 40 mg/L chloride sample, 5 mg/L leading up to and 5 mg/L past the titration endpoint in 1 mg/L intervals. Also illustrated is the silver thiocyanate and silver TPTZ back titration methods, for similar chloride measurement, demonstrating the methods herein of chloride measurement with less harsh or dangerous reagents with a clearer titration endpoint at low chloride concentrations compared to the other current methods that do not use mercury.

The various embodiments described herein thus represent a technical improvement to conventional chloride measurement techniques. Using the techniques as described herein, an embodiment may use an indicator to measure chloride in solution using a method safer for users and more environmentally safe. This is in contrast to the use of dangerous reagents with limitations mentioned above. Such techniques provide a faster and more accurate method for measuring chloride in an aqueous or liquid solution, while using less dangerous or harmful chemicals or reagents in chloride measurement.

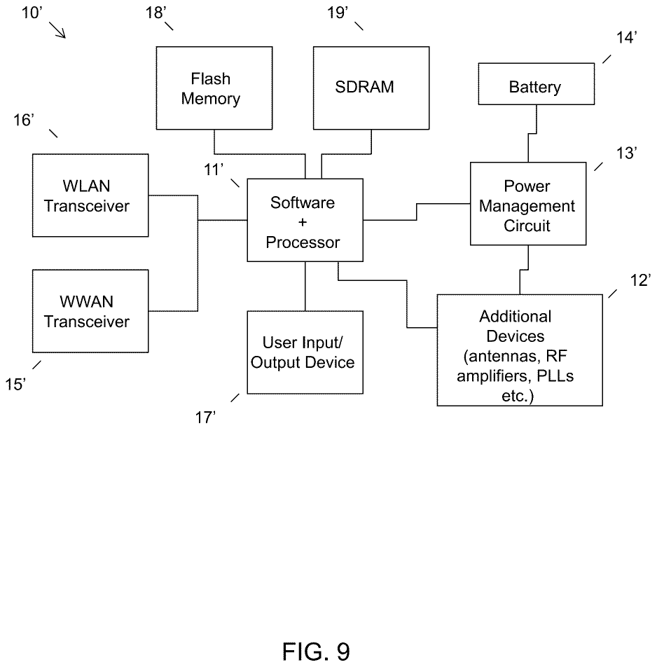

While various other circuits, circuitry or components may be utilized in information handling devices, regarding an instrument for measurement of chloride according to any one of the various embodiments described herein, an example is illustrated in FIG. 9. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment to perform measurement of chloride of an aqueous sample or a sample.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for measuring an amount of chloride in a sample, comprising:
adjusting the pH of the sample to an acidic pH;
introducing an amount of silver nitrate and an amount of iron (II) to the sample to make a reaction solution, wherein the amount of silver nitrate is in excess of the amount of chloride;
back titrating the reaction solution using a 2,4,6-Tripyridyl-s-triazine (TPTZ) solution; and
determining the amount of chloride in the sample from the amount of the titrant used to reach the titration endpoint.

2. The method of claim 1, wherein the adjusting the pH comprises an acetate buffer.

3. The method of claim 1, wherein the acidic pH is a pH range of about 4.0-5.5.

4. The method of claim 1, further comprising mixing the sample, silver nitrate, and the amount of iron (II).

5. The method of claim 1, wherein the back titrating comprises a back titration with the 2,4,6-Tripyridyl-s-triazine (TPTZ) solution to an endpoint in which the reaction solution has a blue color.

6. The method of claim 1, wherein the amount of titrant used is proportional to a concentration of the amount of chloride in the sample.

7. The method of claim 1, wherein the method excludes mercury, dichromate, and thiocyanate.

8. A method for measuring an amount of chloride in a sample, comprising:
adjusting the pH of the sample to an acidic pH;
introducing an amount of silver nitrate, 2,4,6-Tripyridyl-s-triazine (TPTZ), and an amount of iron (II) to make a reaction solution, wherein the amount of silver nitrate is in excess of the amount of chloride;
back titrating the reaction solution using the amount of a chloride solution; and
determining the amount of chloride in the sample from the amount of the titrant used to reach the endpoint.

9. The method of claim 8, wherein the adjusting the pH comprises acetate buffer.

10. The method of claim 8, wherein the acidic pH is a pH range of about 4.0-5.5.

11. The method of claim 8, further comprising mixing the sample, silver nitrate, TPTZ and the amount of iron (II).

12. The method of claim 8, wherein the back titrating comprises a back titration with a chloride solution to an endpoint in which the reaction solution has a blue color.

13. The method of claim 8, wherein the amount of titrant used to reach the endpoint of the titration is proportional to a concentration of the amount of chloride in the sample.

14. The method of claim 8, wherein the method excludes mercury, dichromate, and thiocyanate.

15. A method for measuring an amount of chloride in a sample, comprising:
adjusting the pH of the sample to an acidic pH;
introducing 2,4,6-Tripyridyl-s-triazine (TPTZ) and an amount of iron (II) to the sample to make a reaction solution;
titrating the reaction solution using an amount of a silver nitrate solution; and
determining the amount of chloride in the sample from the amount of the titrant used to reach the endpoint.

16. The method of claim 15, wherein the adjusting the pH comprises acetate buffer.

17. The method of claim 15, wherein the acidic pH is a pH range of about 4.0-5.5.

18. The method of claim 15, wherein the amount of titrant used to reach the endpoint of the titration is proportional to a concentration of the amount of chloride in the sample.

19. The method of claim 15, wherein the titrating comprises a forward titration with a chloride solution to an endpoint in which the reaction solution colorless to yellow from a blue solution.

20. The method of claim 15, wherein the method excludes mercury, dichromate, and thiocyanate.
